(19) United States
(12) Patent Application Publication
(10) Pub. No.: US 2006/0062732 A1
(43) Pub. Date: Mar. 23, 2006

(54) Medicine for detecting lipid components in vivo and vascular endoscope

(75) Inventors: Yasumi Uchida, Funabashi-shi (JP); Haruko Uchida, Funabashi-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Yasumi UCHIDA, Funabashi-shi (JP)

(21) Appl. No.: 11/223,118

(22) Filed: Sep. 12, 2005

(30) Foreign Application Priority Data
Sep. 10, 2004 (JP) 2004-264357
Sep. 14, 2004 (JP) 2004-267281

(51) Int. Cl. A61K 49/00; A61B 10/00

(52) U.S. Cl. 424/9.6; 600/315

(57) ABSTRACT

The present invention provides a medicine and an apparatus which can detect the sites and spreading of in vivo lipids by a two-dimensional image, thereby allowing diagnosis of diseases due to accumulation of lipids.

According to the present invention, provided are a medicine for detecting in vivo lipids, comprising one or more components such as oxLDL and lyso PC, as an effective component, selected from a sulfonic acid blue dye having a radical —SO₃⁻, a homidium, Metanyl yellow and Lissamin fast yellow, and an angioscopic system for obtaining a fluorescent image, which comprises a mercury-xenon lamp light source, a quartz fiberscope wherein the light guide part has 100 to 200 fibers and the image guide part has 7000 to 9000 fibers, a rotational exciting filter, an emitting filter, and a high-sensitive digital camera for picking up the fluorescence as a two-dimensional color image.

### FIELD OF THE INVENTION

The present invention relates to a medicine and an apparatus for detecting lipids existing in a living body (in vivo lipids).

### BACKGROUND OF THE INVENTION

Serious obstructive arterial diseases such as acute coronary syndromes (acute myocardial infarction, unstable angina and sudden cardiac death), cerebral infarction and peripheral arterial obstruction are caused by disruption of atherosclerotic plaques and consequent thrombosis. Lysophosphatidylcholine (lyso PC) is produced by oxidization of phosphatidylcholine (PC), is a chemoattractant and accelerates migration of monocytes and macrophages into the vascular wall (McMurray HF, et al: J Clin Invest 92: 1004-1008, 1993; Yang LV, et al: Blood 105: 1127-1134, 2004; Sonoki K, et al: Metabolism 52: 308-3014, 2003). Oxidized low density lipoprotein (oxLDL) is produced by oxidation of low density lipoprotein (LDL) and accelerates proliferation of macrophages (Sakai M, et al: Atherosclerosis 133: 51-59, 1997). Macrophages in turn become foam cells by accumulating oxLDL into themselves and destruct collagen fibers (Itabe H, et al: J Biol Chem 269: 15278-15281, 1994). These processes induce atherosclerotic plaques vulneable and resultant plaque disruption occurs. Therefore, if lipid components including lyso PC and oxLDL in the vascular wall become visible in vivo, their localization and amount become measurable and the fate of plaques becomes predictable and the effects on them of medical and interventional therapies can be evaluated.

Hitherto, lipoproteins including oxLDL in the blood was measured in vitro by ultracentrifugation (Friedewald W T, et al: Clin Chem 18: 499-502, 1972). Recently, oxLDL is measured as malondialdehyde which is produced by adding thiobarbital (Yagi K: Biochem Med 15: 212-216, 1976) or by ELISA (JP-A-7-238098). All these methods, however, are applicable only in vitro and there are no available tools for measurement and accordingly discrimination of lipid components in the vascular wall or in circulating blood in vivo.

Blue dyes which have —SO₃⁻ (such as Evans Blue) are used for detection of injured vascular endothelial cells (Uchida Y, et al: Am Heart J 130: 1114-1119, 1995). They also inhibit thrombosis (WO 01/93870), vascular restenosis after intervention (WO 01/93871) and migration of progenitor cells into the vascular wall (JP-A-2004-035414). However, it is not known whether blue dyes, yellow dyes such as Methanyl yellow and Lissamin fast yellow, and Homidium evoke fluorescences characteristic of lipid components including oxLDL and lyso PC.

Therefore, it has been undertaken to find out the compounds which evoke fluorescence characteristic of individual lipid components including lyso PC and oxLDL and to establish two dimensional color fluorescent image angioscopy system for detection of fluorescences characteristic of individual lipid components in the vascular wall in vivo.

### SUMMARY OF THE INVENTION

An object of the present invention is to provide a medicine and an apparatus which can discriminate lipids in vivo and detect the kind and spreading of the lipids not by a wavelength but by a two-dimensional color image, thus enabling diagnosis of diseases caused by accumulation of the lipids.

Namely, the present invention relates to a medicine for detecting in vivo lipids, comprising one or more components, as an effective component, selected from a sulfonic acid blue dye having a radical —SO₃⁻, a homidium, Metanyl yellow and Lissamin fast yellow.

Also the present invention relates to a method for detecting in vivo lipids which comprises administering to a subject one or more of components selected from a sulfonic acid blue dye having a radical —SO₃⁻, a homidium, Metanyl yellow and Lissamin fast yellow, and detecting a fluorescence.

In addition, the present invention relates to an angioscopic system (vascular endoscope) for obtaining a fluorescent image comprising a guide catheter having a fiberscope to be inserted into a blood vessel, an excitation unit for exciting a fluorescence and an emission unit for receiving a fluorescent image, said angioscopic system observing, as a two dimensional image, the fluorescence evoked by the substance in the blood vessel when an exciting light is irradiated to the blood vessel; which comprises a mercury-xenon lamp light source, a quartz fiberscope wherein the light guide pait has 100 to 200 fibers and the image guide part has 7000 to 9000 fibers, a rotational exciting filter, an emitting filter, and a high-sensitive digital camera for picking up the fluorescence as a two-dimensional color image.

### BRIEF DESCRIPTION OF THE DRAWINGS

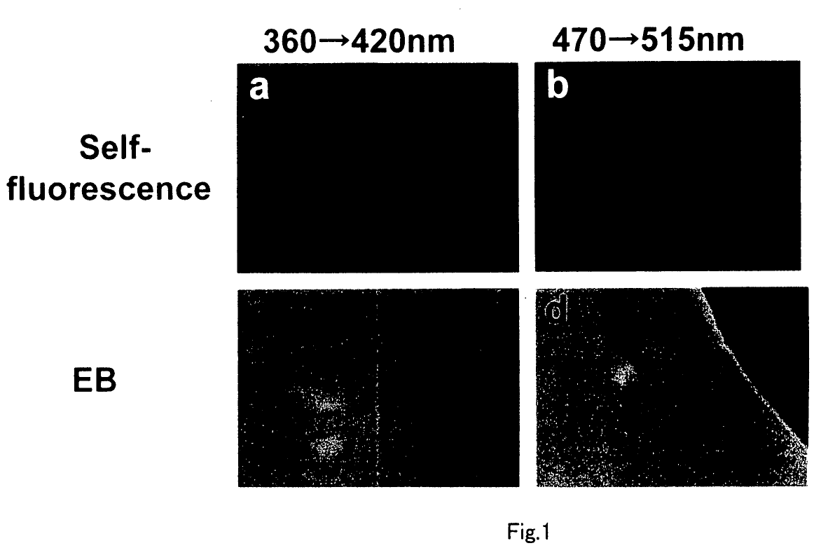

FIG. 1 shows self-fluorescence and fluorescences of oxLDL evoked by Evans blue.
a, b: no self-fluorescence. c: Evans blue-evoked violet fluorescence by exciting at 360 nm and emitting at 420 nm. d: Evans blue-evoked brown fluorescence by exciting at 470 nm and emitting at 515 nm.

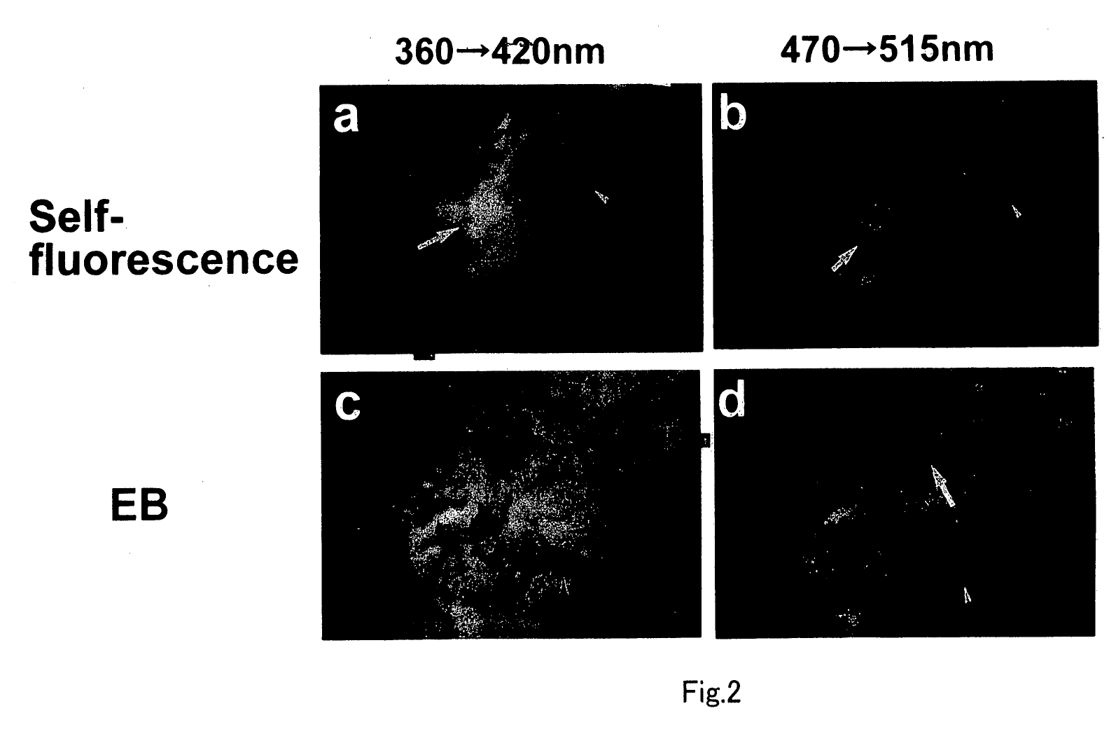

FIG. 2 shows scanning microscopic images of removed human coronary plaque.
s: white self-fluorescence evoked by exciting at 360 nm and emitting at 420 nm (arrow). b: orange self-fluorescence evoked by exciting at 470 nm and emitting at 515 nm (arrow). c: Evans blue-evoked violet fluorescence by exciting at 360 nm and emitting at 420 nm (arrow). d: Evans blue-evoked brown fluorescence by exciting at 470 nm and emitting at 515 nm (arrow). c and d indicate existence of oxLDL.

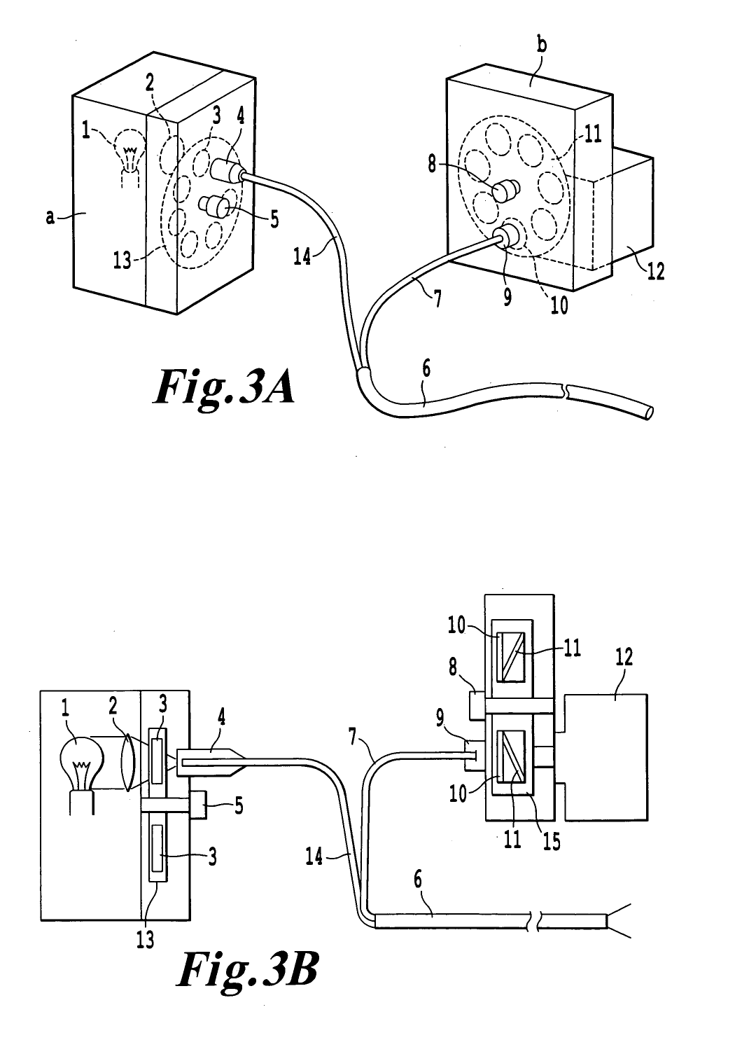

FIG. 3A is a perspective view of the color fluorescent image angioscopic system.
1: Xe—Hg lamp. 2: lens. 3. fluorescence exciting filter. 4: connector to light guide. 5: filter exchange knob. 6: 5F quartz fiberscope. 7: image guide. 8: filter exchange knob. 9: connector of image guide. 10: emitting filter. 11: rotating disk in which exciting filters are incorporated. 12: high sensitive color digital camera. 13: filter rotating disk. 14: light guide, 15: filter plate, a: excitation unit, b: emission unit.

FIG. 3B is a sectional side view of the color fluorescent image angioscopic system.
1: Xe—Hg lamp. 2: lens. 3: fluorescence exciting filter disk. 4: connector to light guide. 5: filter exchange knob. 6: 5F quartz fiberscope. 7: image guide. 8: filter exchange knob. 9: connector of image guide. 10: emitting filter. 11: dichroic filter. 12: high sensitive color digital camera (3CCD digital camera, C7780, Hamamatsu Photo Co, Hamamatsu). 13: filter plate. 14: light guide. 15: filter plate.

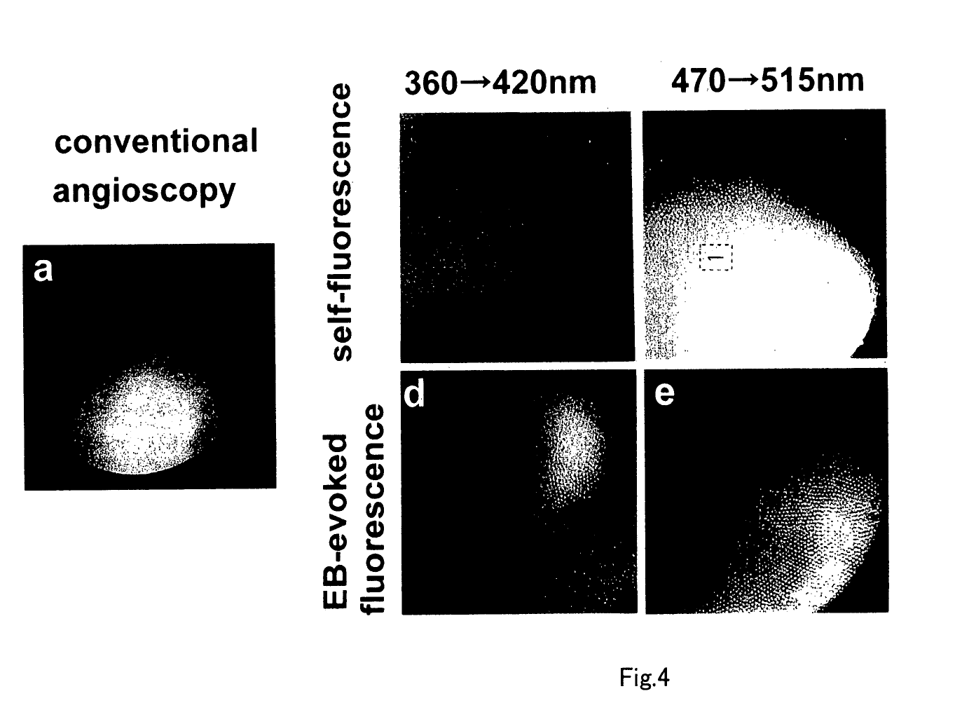

FIG. 4 shows color fluorescent images of a human atherosclerotic coronary plaque visualized by a color fluorescent image angioscopy system.
a: yellow plaque by a conventional fiberscope. b: self-fluorescent image of the plaque by exciting at 360 nm and emitting at 420 nm. Blue fluorescence indicates rich collagen fibers. c: self-fluorescent image of the plaque by exciting at 470 nm and emitting at 515 nm. Yellow fluorescence indicates existence of beta-carotene conjugated with lipids.
d: Evans blue (EB)-evoked fluorescent image of the plaque by exciting at 360 nm and emitting at 420 nm. Violet fluorescence indicates existence of oxLDL.
e: Evans blue (EB)-evoked fluorescent image of the plaque by exciting at 470 nm and emitting at 515 nm. Brown fluorescence indicates existence of oxLDL.

### DETAILED DESCRIPTION OF THE INVENTION

The present inventors have searched for biocompatible substances which have an affinity to lipids such as lipoprotein and have found that a dye having a radical —SO₃⁻ and a homidium conjugate with oxidized LDL, LDL, VLDL, IDL, HDL and components constituting them in vivo to evoke a fluorescence characteristic of the kind thereof. The present inventors have also found that by detecting and observing the fluorescence as an image, the site of the oxidized LDL and the spreading thereof can be known and thus unstable plaques and the like can be diagnosed to complete the present invention.

According to the present invention, it is possible to discriminate and detect lipids such as oxidized LDL, LDL, VLDL, IDL and HDL in vivo. For example, oxidized LDL existing in the vascular wall can be discriminated from the other lipoproteins and detected to enable diagnosis of unstable plaques formed in the vascular wall, whereby it becomes possible to diagnose diseases such as acute myocardial infarction, unstable angina, cerebral infarction and obstructive peripheral arterial sclerosis which are caused by disruption of the unstable plaques, predict onset of these diseases, select a method for treating the diseases, and evaluate the effects of the treatment.

The sulfonic acid blue dye having a radical —SO₃⁻ contained in the medicine according to the present invention may be any substance which emits a characteristic fluorescence when it is conjugated with a lipid in a plaque and is preferably Evans blue, Nile blue or Tripan blue.

As the homidium can be used homidium bromide, homidium chloride, etc.

The blue dye having a radical —SO₃⁻, a homidium, Metanyl yellow and Lissamin fast yellow used in the present invention may be commercial products. The blue dye, a homidium, Metanyl yellow and Lissamin fast yellow may be used singly or as a combination of two or more thereof.

In the medicine for detecting lipids according to the present invention, detectable lipids include, for example, lipoproteins such as oxidized LDL, LDL, VLDL, IDL and HDL, and the components composing the lipoproteins such as cholesterol esters, triglycerides, phosphatidylcholines, oxidized phosphatidylcholines, lyso phosphatidylcholines Qyso PC), free cholesterols, and apolipoprotein B100.

When these lipids are conjugated with the medicine according to the present invention, they evoke a fluorescence characteristic of the kind of the lipids in a specific wavelength region (e.g. 420 nm or 515 nm) by receiving an exciting light of a specific wavelength (e.g. 360 nm or 470 nm) (Tables 2 and 3).

The medicine according to the invention can be formulated into a preparation for parenteral administration or for oral administration in the form of solid or liquid by mixing one or more of the blue dyes, homidiums, Metanyl yellow and Lissamin fast yellow with a pharmaceutically acceptable carrier.

Preparations for parenteral administration include those for intravascular, subcutaneous or intramuscular administration. For preparing them, one or more of the blue dyes, homidiums, Metanyl yellow and Lissamin fast yellow are dissolved in a pharmaceutically acceptable solvent such as physiological saline or an isotonic phosphate buffer and are incorporated with optional ingredients such as propylene glycol, benzyl alcohol and the like, if necessary.

Preparations for oral administration include, for example, solid preparations such as tablets, powders, granules and capsules, and liquid preparations such as solutions, syrups, elixirs, and oily or aqueous suspensions.

Among these administration forms, the medicine according to the invention is preferably used in the form of a preparation for intravascular administration.

The amount of the medicine according to the invention to be used varies depending on the purpose of use, the body part to be applied and the kind of disease, but is generally in a range of approximately from 0.1 to 5 mg/kg.

For detection of lipids in vivo by using the medicine according to the invention, an exciting light of a specific wavelength (for example, in a range of from 330 to 570 nm) is irradiated after a predetermined period of time has elapsed since the medicine was administered, and a fluorescence in a specific wavelength region (for example in a range of from 390 to 660 nm) is detected as a two dimensional color fluorescent image by an angioscopis system.

As the angioscopic system, it is preferable to use the angioscopic system which was found by the present inventors for observing, as a two dimensional image, a fluorescence evoked by the substance in the blood vessel when an exciting light is irradiated to the interior of the blood vessel, and comprises a guide catheter having a fiberscope to be inserted into the blood vessel, an excitation unit for evoking fluorescences, and an emission unit for receiving a fluorescent image; which comprises a mercury-xenon lamp light source, a quartz fiberscope wherein the light guide part has 100 to 200 fibers and the image guide part has 7000 to 9000 fibers, a rotational exciting filter, an emitting filter, and a high-sensitive digital camera for picking up the fluorescence as a two-dimensional color image.

Hitherto, digestive organs and bronchial tubes have been examined by fluorescent shooting using a thick endoscope (JP 8-224208 A). With regard to blood vessels, the size of an endoscope which can be inserted into a blood vessel is at most 1.5 mm in outer diameter. Such an endoscope can supply only very little amount of light, thereby failing to give a color fluorescent image. It was particularly difficult to supply an exciting light in an ultraviolet region to give a color image in a near-ultraviolet region. By using the apparatus according to the present invention, it is possible to pick up a fluorescence in a wide wavelength region as a color image and detect various substances such as collagens and oxidized LDL existing in the vascular walls or in the blood vessel by a color image.

The apparatus will be explained below by way of drawings.

FIG. 3a is a perspective view of the apparatus according to the invention, and FIG. 3b is a side cross-sectional view of the apparatus according to the invention.

The apparatus according to the invention is composed of a guide catheter 6 including a fiberscope to be inserted into blood vessels, an excitation unit a to excite fluorescence, and an emission unit b to receive a fluorescent image (FIG. 3a).

Light source 1 is a light source to generate a light (exciting light) in an ultraviole region and a mercury-xenon lamp is used. A condenser lens 2 for condensing the exciting light generated in the light source 1 is disposed in front of the light source. In front of the condensing lens 2 is disposed a circular filter plate 13 to retain exciting filters 3. A filter plate 13 is incorporated with 7 to 10 different exciting filters 3 which penetrate lights having a wavelength in a range of from 300 nm to 700 nm. By turning a knob 5 for exchanging the exciting filters, a filter plate 13 is rotated and the exciting filter 3 is exchanged. Incidentally, the exciting filter 3 is detachable so tat it can be optionally exchanged for the other filter for the other wavelength. Examples of combinations of the exciting filter 3 and an emitting filter 10 are shown below.

TABLE 1
The wavelength each of the exciting filter and the
corresponding emitting filter

| Exciting filter (nm) | Emitting filter (nm) |
|---|---|
| 320 | 390 |
| 360 | 420 |
| 430 | 475 |
| 470 | 515 |
| 530 | 580 |
| 570 | 610 |
| 660 | 700 |

Note:
The wavelength of the exciting filter is a medium value.

The exciting light having a specific wavelength which has passed through the filter is introduced to the guide catheter 6 including a fiberscope. The fiberscope is composed of a light-guide part 14 connecting the excitation unit a to a vascular cavity and an image guide part 7 connecting the vascular cavity to an emission unit (b). Quartz fibers are used for the fiberscope. The number of the fiber in the light-guide part is preferably 100 to 200, more preferably about 100, and that in the image guide part is preferably 7000 to 9000, more preferably about 8000. By such constitution, a fluorescence in a broad wavelength region can be picked up as a color image.

The guide catheter is preferably a balloon catheter which is equipped with a flexible inserting tube, a balloon integrally connected to the top of the flexible inserting tube, and a channel for pouring physiological saline. The outer diameter of the guide catheter is 1.0 to 1.5 mm.

By irradiating a vascular cavity with an exciting light, a fluorescence is emitted from the substances existing in the vascular wall (for example, lipoproteins, collagens or the like) and is guided to the emission unit b through the image guide part 7. A filter plate 15 similar to the rotating filter plate 13 in the excitation unit a is provided in the emission unit b. The filter plate 15 is incorporated with 7 to 10 different emitting filters 10 which penetrate a fluorescence having a wavelength in a range of from 350 nm to 800 nm. It is preferable to provide at the back of the emitting filters 10 a reflection plate 11 which excludes extra fluorescences.

A fluorescent image which has passed through the emitting filters 10 is received by a high-sensitive color digital camera placed at the back of the filter plate 15. As the high-sensitive color digital camera may be used any camera which can pick up a fluorescent image in a near-ultraviolet region as a two dimensional color image when an exposure time is not less than 100 msec. For example, “3CCD Digital Camera C 7780” (manufactured by Hamamatsu Photonics Corporation) can be used.

In order to detect and diagnose unstable plaques formed on the vascular wall by using the medicine according to the present invention, a preparation for vascular administration is at first prepared by using the medicine according to the present invention, the preparation is then injected into a target artery, and 1 to 5 minutes thereafter, a two dimensional color image is obtained from a fluorescence in a specific wavelength region by utilizing the above-mentioned angioscopic system for obtaining a color (natural color) fluorescent image which includes the guide catheter including the quartz fiberscope to be inserted into blood vessels, the excitation unit for exciting a fluorescence, and the emission unit for receiving a fluorescent image and enables detection of a fluorescent substance by a color, thereby confirming existence of oxidized LDL.

By using the medicine according to the present invention, it is possible to qualify and quantify the lipids in the body liquids including blood by, for example, intravenously administering the medicine according to the present invention and detecting the fluorescence emitted from the blood but not from the vascular wall.

The medicine according to the present invention may be used as a detection reagent to be administered to a disease model of an experimental animal for detecting unstable plaques formed in the vascular wall and studying the relation between the unstable plaques and various diseases, thereby providing medically useful information.

### EXAMPLES

### Example 1

Microscopic Examination of Fluorescences Characteristic of Major Substances Composing Atherosclerotic Plaques

Self-fluorescences and dye-evoked fluorescences of the major substances composing the atherosclerotic plaque were examined by fluorescent microscopy.

1) Materials and Methods

a) Commercially obtainable substances composing atherosclerotic plaques were added to one of the dyes and the evoked fluorescences were examined by fluorescent microscopy (Olympus). The substances used are as follows: oxidized low density lipoprotein (oxLDL; Biogenesis LTd, Kingston), low density lipoprotein (LDL; Carbochem Co, LaJolla), very low densitiy lipoprotein (VLDL; Athens Res & Tech, Athens), intermediate density lipoprotein (IDL; personally isolated), high density lipoprotein (HDL; Chemicon Int.), phosphatidylcholine (PC; Sigma Co, St. Louis), L(a)-lyso phosphatidylcholine (lyso PC; Sigma Co, St. Louis), apolipoprotein B100 (apoB 100; ICN Biomedicals, Ohio), Triglyceride (TG; Serdary Res. Lab., Canada), free Cholesterol (Wako, Osaka), Cholesteryl linolate (Doosan Serdary Res. Lab., Toronto), Heparan sulfate (Sigma Co, St. Louis), Hyaluronic acid (Sigma Co, St. Louis), Collagen I, II, III, IV, VI (Sigma Co., St. Louis), Calcium phosphate tribasic (Wako, Osaka), beta-Carotene (Ohtsuka Co, Tokyo), Evans blue (Sigma Co., St. Loius), Nile blue (E. Merck, Darmstadt), Tripan blue (Sigma Co., St. Louis), Methanyl yellow (Wako, Osaka), Lissamin fast yellow (Research Chem, Lancs), Homidium chloride (Wako, Osaka).

b) Self-fluorescence

Water-soluble substances were diluted in distilled water and 0.05 ml was mounted on deck glass and self-fluorescences were examined by exciting at 360 nm and emitting at 420 nm or by exciting at 470 nm and emitting at 515 nm using microscope (Olympus Co. model 100) at ×40. Water insoluble substances such as TG and beta-Carotene were dissolved in other solvents and were examined similarly. Other substances such as free cholesterol, cholesteryl linolate, calcium phosphate and collagens were examined as powder, crystal or fiber (Table I). Dyes were dissolved in distilled water (10⁻⁵ M) and 0.05 ml of them were mounted on deck glass and were examined similarly (Table I).

c) Dye-evoked fluorescences of major substances composing atherosclerotic plaque

0.05 ml of one of the dye solutions was mounted on deck glass and then 0.05 ml solution of one of the soluble substances composing the plaque was added and dye-evoked fluorescence was examined by exciting at 360 nm and emitting at 420 nm or by exciting at 470 nm and emitting at 515 nm. In case of free cholesterol, cholesteryl linolate and calcium phosphate, 0.1 mg of powder, crystal or fiber was added to the dye solution and dye-evoked fluorescence was examined similarly.

2) Results

a) As summarized in Table 2, except Homidium, Evans blue, Nile blue, Tripan blue, Metanyl yellow and Lissamine fast yellow had no self-fluorescence.

PC, free cholesterol, cholesteryl linolate, calcium phosphate, collagens and beta-carotene had self-fluorescence but the other substances did not. Blue fluorescence by exciting at 360 nm and emitting at 420 nm was characteristic of collagens.

TABLE 2
Self-fluorescence of Dyes and Major Substances
Composing Atherosclerotic Plaques

| | | Exciting | |
|---|---|---|---|
| | | filter (nm) | |
| | | 360 | 470 |
| | | Emitting | |
| | | filter (nm) | |
| | Concentration | 420 | 515 |
| Evans blue | (10⁻⁵ M) | | |
| Nile blue | (10⁻⁵ M) | | |
| Tripan blue | (10⁻⁵ M) | | |
| Metanyl yellow | (10⁻⁵ M) | | |
| Lissamine | (10⁻⁵ M) | | |
| Homidium chloride | (10⁻⁵ M) | Y | O |
| OxLDL | (0.1 mg/ml) | | |
| LDL | (0.1 mg/ml) | | |
| VLDL | (0.1 mg/ml) | | |
| ILDL | (0.1 mg/ml) | | |
| HDL | (0.1 mg/ml) | | |
| PC | (0.1 mg/ml) | W | |
| Lyso PC | (0.1 mg/ml) | | |
| ApoB100 | (0.1 mg/ml) | | |
| TG | (2 mg/ml hexane) | | |
| free Cholesterol | (powder) | W | Y |
| Cholesteryl linolate | (powder) | W | Y |
| Heparan sulfate | (0.1 mg/ml) | | |
| Calcium phosphate | (crystal) | W | Y |
| Collagen I, II, III, IV, VI | (fiber) | B | G |
| beta-Carotene | (0.1 mg/ml, glycerin) | O | O |

Lissamine: Lissamine fast yellow.
Unfilled columns indicate no self-fluorescence.
B: blue.
G: green.
O: orange.
W: white.
Y: yellow.

b) Dye-evoked fluorescence

FIG. 1 shows Evans blue-evoked violet fluorescence by exciting at 360 nm and emitting at 420 nm and brown fluorescence by exciting at 470 nm and emitting at 515 nm. This combination of color fluorescence was characteristic of oxLDL (FIG. 1). Evans blue also evoked color fluorescences characteristic of HDL, cholesteryl linolate and hyaluronic acid (underlined fluorescent colors in Table 3). Nile blue evoked color fluorescence characteristic of oxLDL and TG. Tripan blue evoked color fluorescence characteristic of lyso PC, calcium phosphate and heparan sulphate. Metanyl yellow and Lissamine fast yellow evoked color fluorescence characteristic of lyso PC (Table 3).

Homidium has self-fluorescence but it evoked different color fluorescence when added to the substances composing the plaque. This dye evoked red fluorescence characteristic of lyso PC by exciting at 360 nm and emitting at 420 nm and by exciting at 470 nm and emitting at 515 nm. Although Tripan blue masked Homisium-evoked fluorescence of several substances but it did not influence that of lyso PC (“Table 4).

TABLE 3
Dye-evoked Fluorescences of Major Substances Composing Atherosclerotic
Plaques

| | EB | | NB | | TB | | Metanyl | | Lissamine | |
|---|---|---|---|---|---|---|---|---|---|---|
| | Exciting filter (nm) | | | | | | | | | |
| | 360 | 470 | 360 | 470 | 360 | 470 | 360 | 470 | 360 | 470 |
| | Emitting filter (nm) | | | | | | | | | |
| | 420 | 515 | 420 | 515 | 420 | 515 | 420 | 515 | 420 | 515 |
| OxLDL | V | Br | | Go | V | O | | Y | | |
| LDL | | R | O | O | V | O | | | | |
| VLDL | | R | | G | V | R | | | O | O |
| IDL | | O | | G | | G | | | Y | R |
| HDL | R | R | | G | P | R | | | O | O |
| PC | V | O | R | O | P | O | Br | R | W | Y |
| Lyso PC | V | O | R | O | R | R | Br | Br | Br | Br |
| ApoB100 | O | O | O | G | P | O | | | | |
| TG | | | Y | Y | P | O | | | | |
| Cholesteryl | B | G | | | | | | | | |
| Linolate | | | | | | | | | | |
| Free | | O | | | | | | | | |
| Cholesterol | | | | | | | | | | |
| Collagen I-VI | | | | | | | | | | |
| Calcium | B | Y | | | B | G | | | | |
| phosphate | | | | | | | | | | |
| Hyaluronic | SB | | | | | | | | | |
| acid | | | | | | | | | | |
| Heparan | | | | | | | | | | |
| Sulfate beta- | | | | | W | G | | | | |
| Carotene | | | | | | | | | | |

EB: Evans blue.
NB: Nile blue.
TB: Tripan blue.
Metanyl: Metanyl yellow.
Lisammine: Lisammine fast yellow.
oxLDL: oxidized low density lipoprotein.
LDL: low density lipoprotein.
VLDL: very low density lipoprotein.
IDL: intermediate density lipoprotein.
PC: phosphatidylcholine.
Lyso PC: lysophosphatidyl choline.
ApoB100: apolipoprotein B100.
TG: triglyceride.
B: blue.
Br: brown.
G: green.
Go: gold.
O: orange.
P: purple.
R: red.
SB: sky blue.
V: violet.
W: white.
Y: yellow.
Underlined fluorescence is characteristic of the corresponding substance.
Unfilled columns indicate no fluorescence.

TABLE 4
Fluorescences of the Major Substances Composing
Atherosclerotic Plaques Evoked by Homidium
Chloride and Tripan Blue

| | |
|---|---|
| Homidium | Homidium +TB |

| Exciting filter (nm) | 360 | 470 | 360 | 470 |
|---|---|---|---|---|
| Emitting filter (nm) | 420 | 515 | 420 | 515 |
| OxLDL | V | O | Y | Go |
| LDL | P | O | V | Go |
| VLDL | P | O | V | O |
| IDL | P | O | V | O |
| HDL | P | Y | V | O |
| PC | P | Y | P | O |
| Lyso PC | R | R | R | R |
| ApoB | RO | RO | RO | RO |
| TG | O | O | | |
| Cholesteryl Linolate | W | Y | | |
| free cholesterol | W | Y | W | G |
| Collagen I-VI | P | O | | |
| Calcium Phosphate | W | Y | | |
| Hyaluronic acid | | | | |
| Heparan Sulfate | | | | |
| beta-carotene | O | O | P | O |

RO: reddish orange.

3) Conclusion

Evans blue and Nile blue evoke color fluorescence characteristic of oxLDL. Tripan blue, Metanyl yellow and Lissamibn fast yellow evoke color fluorescence characteristic of lyso PC.

### Example 2

Scanning Microscopic Observation of Dye-evoked Fluorescences Characteristic of Lipid Components in the Atherosclerotic Plaques of Human Coronary Artery

1) Materials and Methods

Atherosclerotic plaques of coronary artery removed from human cadavers by permission of family was cut into 2×2 mm² segments, dipped into 10⁻⁵ M dye solution for 5 min, and they were washed with saline to mounted on deck glass for fluorescent microscopic observation.

2) Results

FIG. 2 shows Evans blue-evoked color fluorescence of human coronary plaque. Before dye administration, the plaque exhibited white to yellow self-fluorescence (arrow in a) by exciting at 360 nm and emitting at 420 nm and yellow to orange fluorescence by exciting at 470 nm and emitting at 515 nm (arrow in b), indicating existence of calcium phosphate, free cholesterol and beta-Carotene. After Evans blue administration, violet fluorescence was evoked in the plaque (arrow in c) by exciting at 360 nm and emitting at 420 nm and brown fluorescence by exciting at 470 and emitting at 515 nm (arrow in d) indicating existence of oxLDL. Nile blue evoked golden fluorescence by exciting at 470 and emitting at 515 nm, indicating existence of oxLDL. Tripan blue and Homidium evoked red fluorescence by exciting at 360 nm and emitting at 420 nm, and by exciting at 470 nm and emitting at 515 nm, indicating existence of lyso PC.

3) Conclusion

oxLDL and lyso PC were detected by fluorescent microscopy in the removed human coronary plaques.

### Example 3

Observation of Dye-evoked Fluorescence Characteristic of Lipid Components in Atherosclerotic Plaques of Human Coronary Artery by Color Fluorescent Image Angioscopy System

1) Materials and Methods

a)Color fluorescent image angioscopy system

The color fluorescent image angioscopy system is composed of a fiberscope with 200 quartz fibers for light guide and 7000 quartz fibers for image guide incorporated in a 5 F balloon guiding catheter, fluorescence exciting part, fluorescence emitting part, 3CCD color digital camera (C37780, Hamamatsu Photo, Co, Hamamatsu), DVD and monitor. The exciting part is composed of Xe—Hg lamp, lens, and 7 exciting filters imbedded in a rotating disk. The emitting part is composed of 7 emitting filters imbedded in a rotating disk (FIG. 3). The wavelength of the exciting and emitting filters is shown in Table IV. By changing filters by rotation of the disks, a wide range of fluorescent wavelength can be examined.

b) Perfusion circuit

The epicardial coronary arteries removed from human cadavers by permission of family were used. Five F polyethylene tubes were inserted into the proximal and distal ends of the artery, and a Y connecter was connected to the proximal end. A polyethylene tube was connected to the side hole of Y connector to perfuse saline at 1 ml/sec by a pump. A fiberscope was introduced through the Y connecter into the artery. At first, the luminal surface of the artery were surveyed by a conventional angioscopy system for detection of plaques (Uchida Y: Coronary Angioscopy, Futura Pub Co, NY, 2001). Then, two dimensional color self-fluorescent images of the plaque were examined by exciting at 360 nm and emitting at 420 nm or by exciting at 470 nm and emitting at 515 nm. Thereafter, the artery was perfused with one of the dye solution (10-5 M) for 5 min. and the dye solution was changed to saline for observation.

TABLE 5
Exciting filters and Their Corresponding Emitting Filters
of Color Fluorescence Filters of the Fluorescent Image
Angioscopy System

| Exciting filter (+15 nm) | Emitting filter (nm) |
|---|---|
| 360 | 420 |
| 430 | 475 |
| 470 | 515 |
| 530 | 580 |
| 570 | 610 |
| 660 | 700 |

2) Results

FIG. 4 shows Evans blue evoked fluorescent images of a coronary plaque. The plaque was yellow in color by a conventional angioscopy (a). Before Evans blue, the plaque exhibited blue fluorescence by exciting at 360 nm and emitting at 420 nm, and yellow fluorescence by exciting at 470 nm and emitting at 515 nm, indicating existence of collagens and beta-Carotene. After Evans blue Perfusion, the plaque exhibited violet fluorescence by exciting at 360 nm and emitting at 420 nm, and brown fluorescence by exciting at 470 nm and emitting at 515 nm, indicating existence of oxLDL. Similarly, golden fluorescence was observed after perfusion with Nile blue solution, indicating existence of oxLDL, red fluorescence after perfusion with Tripan blue or Homidium chloride, indicating existence of lyso PC, and brown fluorescence after perfusion with Metanyl yellow or Lissamine fast yellow solution, indicating existence of lyso PC.

3) Conclusion

The results indicate that oxLDL and lyso PC in the human atherosclerotic plaques are detectable by a color fluorescent image angioscopy system in vivo.

1. A medicine for detecting in vivo lipids, comprising one or more components, as an effective component, selected from a sulfonic acid blue dye having a radical —SO₃⁻, a homidium, Metanyl yellow and Lissamin fast yellow.

2. The medicine according to claim 1, which is for detecting an oxidized low density lipoprotein and lyso PC in plaques.

3. The medicine according to claim 1 or 2, wherein the sulfonic acid blue dye is Evans Blue, Nile Blue or Tripan Blue.

4. A method for detecting in vivo lipids which comprises administering one or more of components selected from a sulfonic acid blue dye having a radical —SO₃⁻, a homidium, Metanyl yellow and Lissamin fast yellow, and detecting a fluorescence.

5. The method according to claim 4, wherein the in vivo lipid is an oxidized low density lipoprotein and lyso PC in plaques.

6. An angioscopic system for obtaining a fluorescent image and comprising a guide catheter having a fiberscope to be inserted into a blood vessel, an excitation unit for exciting a fluorescence and an emission unit for receiving a fluorescent image, said angioscopic system observing, as a two dimensional image, the fluorescence evoked by the substance in the blood vessel when an exciting light is irradiated to the blood vessel; which comprises a mercury-xenon lamp light source, a quartz fiberscope wherein the light guide part has 100 to 200 fibers and the image guide part has 7000 to 9000 fibers, a rotational exciting filter, an emitting filter, and a high-sensitive digital camera for picking up the fluorescence as a two-dimensional color image.

7. The angioscopic system according to claim 6, wherein the transmission wavelength of the exciting filter is in a range of from 300 nm to 700 nm, and the transmission wavelength of the emitting filter is in a range of from 350 nm to 800 nm.
